(19) United States
(12) Patent Application Publication
(10) Pub. No.: US 2015/0273231 A1
(43) Pub. Date: Oct. 1, 2015

(54) PLASMA SYSTEM

(71) Applicant: Electronics and Telecommunications Research Institute, Daejeon (KR)

(72) Inventors: Yark Yeon KIM, Daejeon (KR); Han Young YU, Daejeon (KR); Won Ick JANG, Daejeon (KR); Yong Sun YOON, Daejeon (KR); Bong Kuk LEE, Daejeon (KR)

(21) Appl. No.: 14/634,416

(22) Filed: Feb. 27, 2015

(30) Foreign Application Priority Data
Mar. 31, 2014 (KR) 10-2014-0038063
Nov. 7, 2014 (KR) 10-2014-0154526

(51) Int. Cl. A61N 1/44; H05H 1/24

(52) U.S. Cl. CPC A61N 1/44; H05H 1/2406; H05H 2001/2443

(57) ABSTRACT

Provided herein is a plasma system including a nozzle including an outer circumference exposed towards outside, an inner circumference facing the outer circumference and touching gas, and an exit from which the gas is sprayed; a first electrode formed on a portion of the outer circumference or inner circumference; and a second electrode formed on a portion of the outer circumference and distanced from the first electrode; wherein the first electrode is electrically connected to a first power having a first voltage, and the second electrode is electrically connected to a second power having a second voltage that is different from the first voltage, and the second electrode is formed closer to the exit than the first electrode.

### CROSS-REFERENCE TO RELATED APPLICATION

The present application claims priority to Korean patent application numbers 10-2014-0038063 filed on Mar. 31, 2014 and 10-2014-0154526 filed on Nov. 7, 2014, the entire disclosure of which is incorporated herein in its entirety by reference.

### BACKGROUND

1. Field of Invention

Various embodiments of the present disclosure relate to a low temperature atmospheric large area plasma system for skin care and medical use, and more particularly, to a low temperature atmospheric large area plasma for skin care and medical use.

2. Description of Related Art

Due to the thermal characteristics of plasma, low temperature atmospheric large area plasma had been applied to medical fields until the early 2000s such as to coagulate blood or remove a tissue during operation. And since the early 2000s, low temperature atmospheric large plasma has been widely used in apparatuses such as air purifiers and harmful gas filters due to the properties of plasma of sterilizing and disinfecting microbes. Furthermore, in recent years, new apparatuses are drawing attention based on research results on the interactions between plasma and living body cells.

In order to use a low temperature atmospheric plasma system as a medical device, there is a need for a variety of structures depending on the application field together with the stability against temperature. There are two types of plasma systems currently being studied and developed. First is an indirect plasma method that does not bring a plasma plume into direct contact with the skin, wherein plasma is generated far away from an area to be treated and the generated plasma is then guided to the area by a carrier gas (inert gas). A disadvantage of this method is that it is less effective in treating the area. Second is a direct plasma method of directing bringing a plasma plume into direct contact with an area to be treated, that is, a technology that is based on a plasma jet. This method is highly effective in terms of treatment but can only be applied to small local areas.

When using the aforementioned method of generating plasma and then guiding the generated plasma by a carrier gas, a high voltage is required in order to form a uniform low temperature plasma over a wide area under an atmospheric pressure. To lower such a high breakdown voltage, helium (He) that has a low breakdown voltage and a high heat conductivity is used as carrier gas, but helium is not economically desirable since it costs a lot. Therefore, there is a need for a plasma system that generates plasma at a low voltage using argon (Ar) that is inexpensive as carrier gas, and an efficient electrode design for the system.

### SUMMARY

Various embodiments of the present disclosure are directed to a direct plasma type atmospheric system for medical use that is capable of obtaining an efficient treatment effect over a wide area.

Furthermore, various embodiments of the present disclosure are directed to a plasma system configured to form a uniform low temperature plasma brush over a wide area under an atmospheric pressure while applying a low voltage using preionization.

One embodiment of the present disclosure provides a plasma system including a nozzle comprising an outer circumference exposed towards outside, an inner circumference facing the outer circumference and touching gas, and an exit from which the gas is sprayed; a first electrode formed on a portion of the outer circumference or inner circumference; and a second electrode formed on a portion of the outer circumference and distanced from the first electrode; wherein the first electrode is electrically connected to a first power source having a first voltage, and the second electrode is electrically connected to a second power source having a second voltage that is different from the first voltage, and the second electrode is formed closer to the exit than the first electrode.

The present disclosure provides a direct plasma type atmospheric pressure plasma system for medical use that is capable of obtaining an efficient treatment effect over a wide area.

Furthermore, the present disclosure provides a plasma system configured to form a uniform low temperature plasma brush over a wide area under an atmospheric pressure while applying a low voltage using preionization.

### BRIEF DESCRIPTION OF THE DRAWINGS

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey the scope of the example embodiments to those skilled in the art.

In the drawing figures, dimensions may be exaggerated for clarity of illustration. It will be understood that when an element is referred to as being “between” two elements, it can be the only element between the two elements, or one or more intervening elements may also be present. Like reference numerals refer to like elements throughout.

FIG. 1 is a perspective view for explaining a plasma system according to an embodiment of the present disclosure;

FIG. 2a illustrates a front view and side view for explaining a plasma system according to an embodiment of the present disclosure;

FIG. 2b is a side view for explaining a plasma system according to another embodiment of the present disclosure;

FIG. 2c is a cross-sectional view cut along A-A′ for explaining a plasma system according to another embodiment of the present disclosure;

FIG. 3a is a perspective view for explaining a plasma system according to another embodiment of the present disclosure;

FIG. 3b is a side view for explaining a plasma system according to another embodiment of the present disclosure;

FIG. 3c is a cross-sectional view cut along B-B′ for explaining a plasma system according to another embodiment of the present disclosure;

FIG. 4a is a perspective view for explaining a plasma system according to another embodiment of the present disclosure;

FIG. 4b is a side view for explaining a plasma system according to another embodiment of the present disclosure;

FIG. 4c is a cross-sectional view cut along C-C′ for explaining a plasma system according to another embodiment of the present disclosure; and

FIGS. 5 and 6 views for explaining a pulse power source of a plasma system according to another embodiment of the present disclosure.

### DETAILED DESCRIPTION

Hereinafter, embodiments will be described in greater detail with reference to the accompanying drawings. Embodiments are described herein with reference to cross-sectional illustrations that are schematic illustrations of embodiments (and intermediate structures). As such, variations from the shapes of the illustrations as a result, for example, of manufacturing techniques and/or tolerances, are to be expected. Thus, embodiments should not be construed as limited to the particular shapes of regions illustrated herein but may include deviations in shapes that result, for example, from manufacturing. In the drawings, lengths and sizes of layers and regions may be exaggerated for clarity. Like reference numerals in the drawings denote like elements.

Terms such as ‘first’ and ‘second’ may be used to describe various components, but they should not limit the various components. Those terms are only used for the purpose of differentiating a component from other components. For example, a first component may be referred to as a second component, and a second component may be referred to as a first component and so forth without departing from the spirit and scope of the present disclosure. Furthermore, ‘and/or’ may include any one of or a combination of the components mentioned.

Furthermore, a singular form may include a plural from as long as it is not specifically mentioned in a sentence. Furthermore, “include/comprise” or “including/comprising” used in the specification represents that one or more components, steps, operations, and elements exist or are added.

Furthermore, unless defined otherwise, all the terms used in this specification including technical and scientific terms have the same meanings as would be generally understood by those skilled in the related art. The terms defined in generally used dictionaries should be construed as having the same meanings as would be construed in the context of the related art, and unless clearly defined otherwise in this specification, should not be construed as having idealistic or overly formal meanings.

It is also noted that in this specification, “connected/coupled” refers to one component not only directly coupling another component but also indirectly coupling another component through an intermediate component. On the other hand, “directly connected/directly coupled” refers to one component directly coupling another component without an intermediate component.

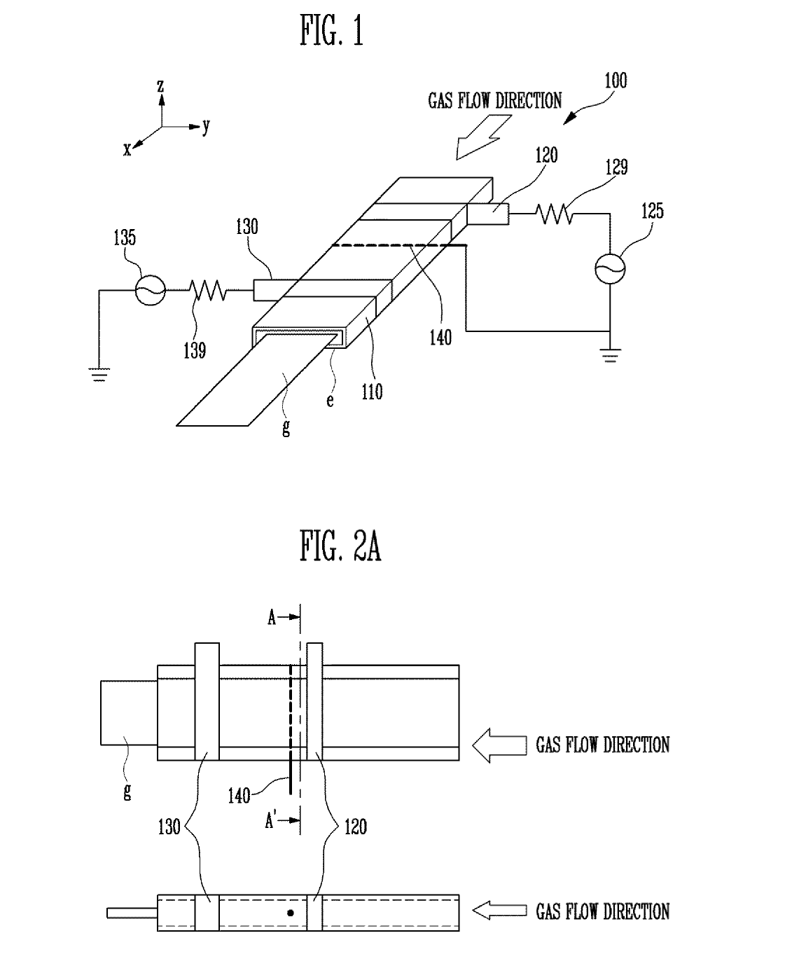

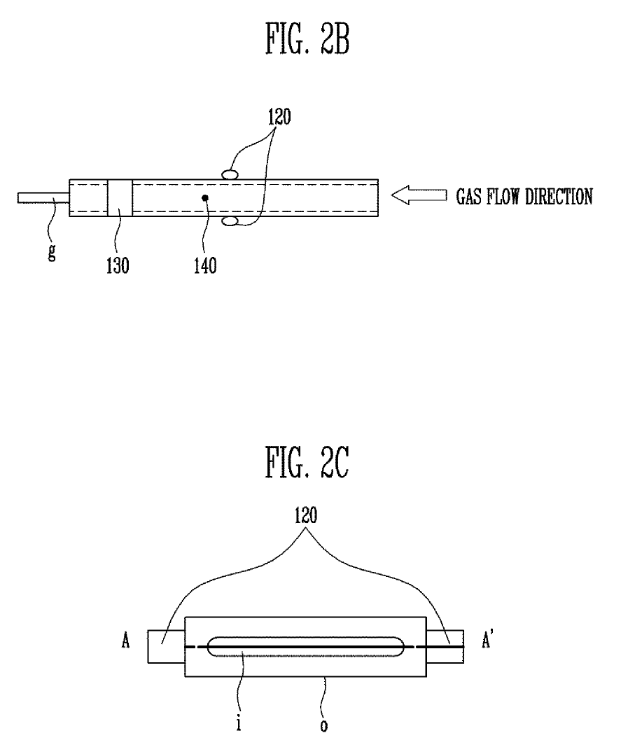

FIG. 1 is a perspective view for explaining a plasma system according to an embodiment of the present disclosure; FIG. 2a illustrates a front view and side view for explaining a plasma system according to an embodiment of the present disclosure; FIG. 2b is a side view for explaining a plasma system according to another embodiment of the present disclosure; and FIG. 2c is a cross-sectional view cut along A-A′ for explaining a plasma system according to another embodiment of the present disclosure. Referring to FIGS. 1, 2a, 2b, and 2c, the plasma system 100 includes a nozzle 110, first electrode 120, second electrode 130, and third electrode 140.

The nozzle 110 includes an outer circumference (o), an inner circumference (i) facing the outer circumference (o) and touching carrier gas (g), and an exit (e) from which the carrier gas (g) is sprayed.

The first electrode 120 is an electrode for preliminary ionization(preionization), and is formed on a portion of the outer circumference (o) and is connected to a first power source 125 through a first resistance 129. A second electrode 130 is an electrode for ionization, and is formed on a portion of the outer circumference (o) such that it is distanced from the first electrode 120, and is connected to the second power source 135 through a second resistance 139. The third electrode 140 is a ground electrode, and is formed on a portion of the inner circumference (i) facing the outer circumference (o), and is electrically connected to a ground of the first power source 125. The first resistance 129 and second resistance 139 may desirably be ballast resistances so as to improve the stability of the preliminary ionization, and each of the first power source 125 and second power source 135 may be an alternating current, bipolar pulse, unipolar pulse, or direct current.

The first electrode 120 may have a structure wherein a conductor tape or conductor wire is wound around the outer circumference (o) as illustrated in FIG. 2a, or a structure wherein two conductor wires are arranged on two surfaces (for example, upper surface and lower surface) facing each other as illustrated in FIG. 2b. The second electrode 130 is formed on a portion of the outer circumference (o), and thus may have a similar structure as the first electrode 120. The third electrode 140 may have a structure of a conductor wire adhered to a portion of the inner circumference (i), or two conductor wires adhered to two different portions on the inner surface (i) as illustrated in FIG. 2c. As illustrated in FIG. 2c, the inner circumference (i) is processed to be round in consideration of a friction with the carrier gas, and the nozzle 110 may desirably be made of an insulator material.

The carrier gas (g) is preliminary-ionized(preionized) by the first electrode 120 and third electrode 140 as it flows towards the exit. The shorter a distance between the first electrode 120 and third electrode 140, the smaller the size of voltage level necessary for generating plasma. A plume generated due to the preliminary ionization (preionization) flows towards the exit (e). Furthermore, ions and electrons generated due to the preliminary ionization may be induced to the second electrode 130 by the carrier gas. When the second electrode 130 is ionized, plumes, ions, and electrons generated by the first electrode 120 play the role of seeds, and thus even when a low voltage level is applied to the second electrode 130, plasma may be generated. Therefore, even when the voltage level of the second electrode 130 is relatively low, a plume may be maintained even where it is far away from the exit (e) (the length and area formed by the plume increase).

A plasma system according to an embodiment of the present disclosure uses preliminary ionization of a dielectric barrier discharge (DBD), and thus may generate a uniform and stable plasma even at a low voltage. Furthermore, the plasma system according to the present disclosure 100 may form a plasma plume over a wide area even when power having a low voltage level is applied. Furthermore, the uniformity of the plasma plume may be improved and the length of the plume formed in the nozzle may be increased. Therefore, the plasma system according to the embodiment of the present disclosure has an effect that it may be used more efficiently in skin wounds and diseases of various sizes and that various gases may be used locally for treatment. Furthermore, based on such a technology, the plasma system is applicable to treatments of wounds of wider areas such as in skin care and diseases.

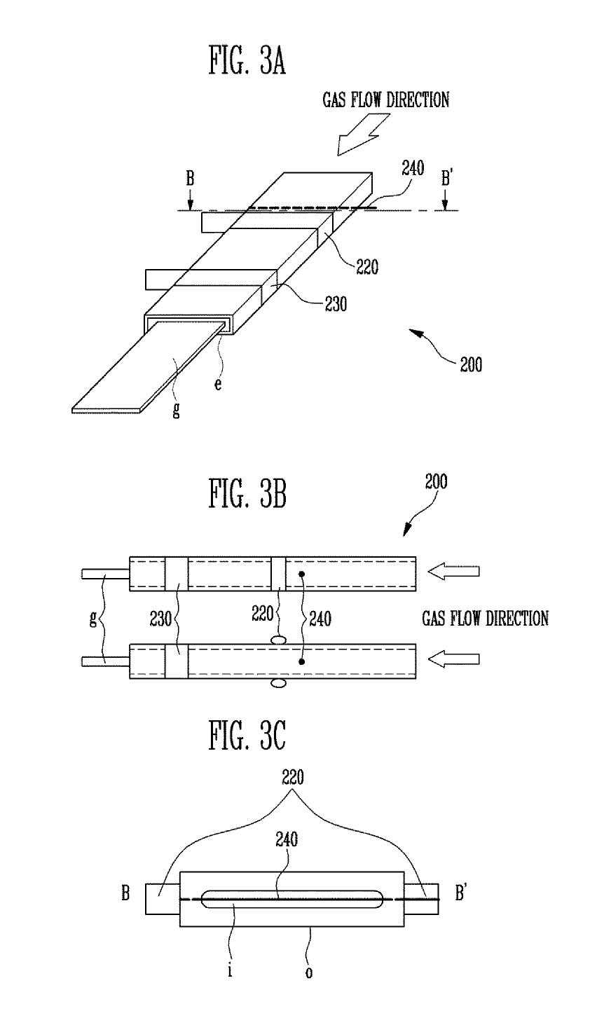

FIG. 3a is a perspective view for explaining a plasma system according to another embodiment of the present disclosure, FIG. 3b is a side view for explaining a plasma system according to another embodiment of the present disclosure, and FIG. 3c is a cross-sectional view for explaining a plasma system according to another embodiment of the present disclosure.

Referring to FIGS. 3a to 3c, a plasma system 200 includes a nozzle 210, first electrode 220, second electrode 230, and third electrode 240. In the embodiment illustrated in FIG. 1, the distance between the third electrode 140 and exit (e) is shorter than the distance between the first electrode 120 and exit (e), but longer than the distance between the second electrode 130 and exit (e). However, in the embodiment illustrated in FIG. 3a, the distance between the third electrode 240 and exit (e) is longer than the distance between the first electrode 220 and exit (e), and the distance between the second electrode 230 and exit (e).

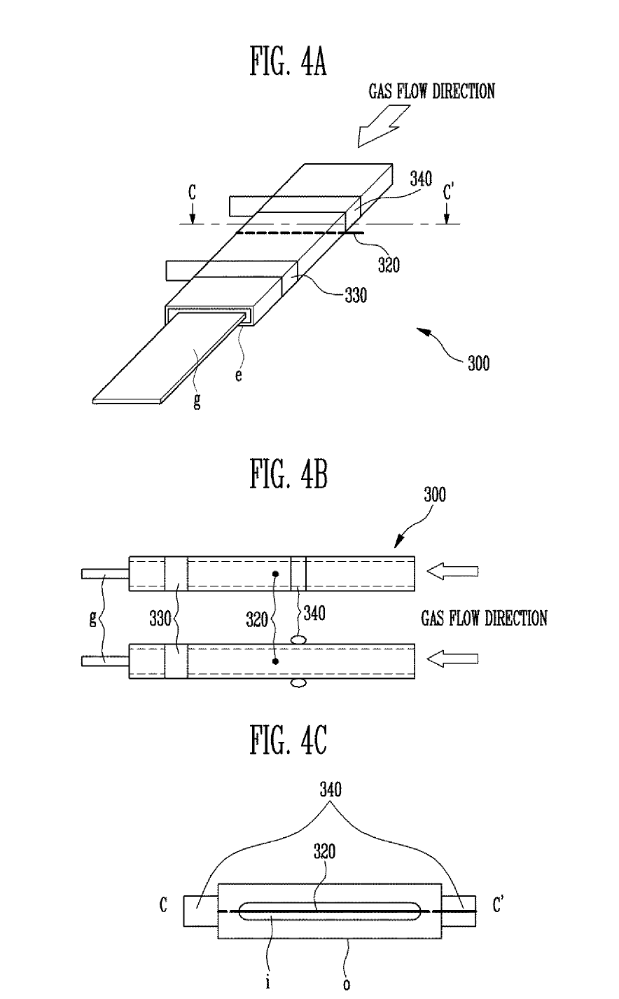

FIG. 4a is a perspective view for explaining a plasma system according to another embodiment of the present disclosure, FIG. 4b is a side view for explaining a plasma system according to another embodiment of the present disclosure, and FIG. 4c is a cross-sectional view cut along C-C′ for explaining a plasma system according to another embodiment of the present disclosure.

Referring to FIGS. 4a to 4c, a plasma system 300 includes a nozzle 310, first electrode 320, second electrode 330, and third electrode 340. In the embodiment illustrated in FIG. 1, the first electrode 120 is formed on a portion of the outer circumference (o), and the third electrode 140 is formed on a portion of the inner circumference (i) facing the outer circumference (o). However, in the embodiment illustrated in FIG. 4c, the first electrode 320 is formed on a portion of the inner circumference (i), and the third electrode 340 is formed on a portion of the outer circumference (o) facing the inner circumference (i).

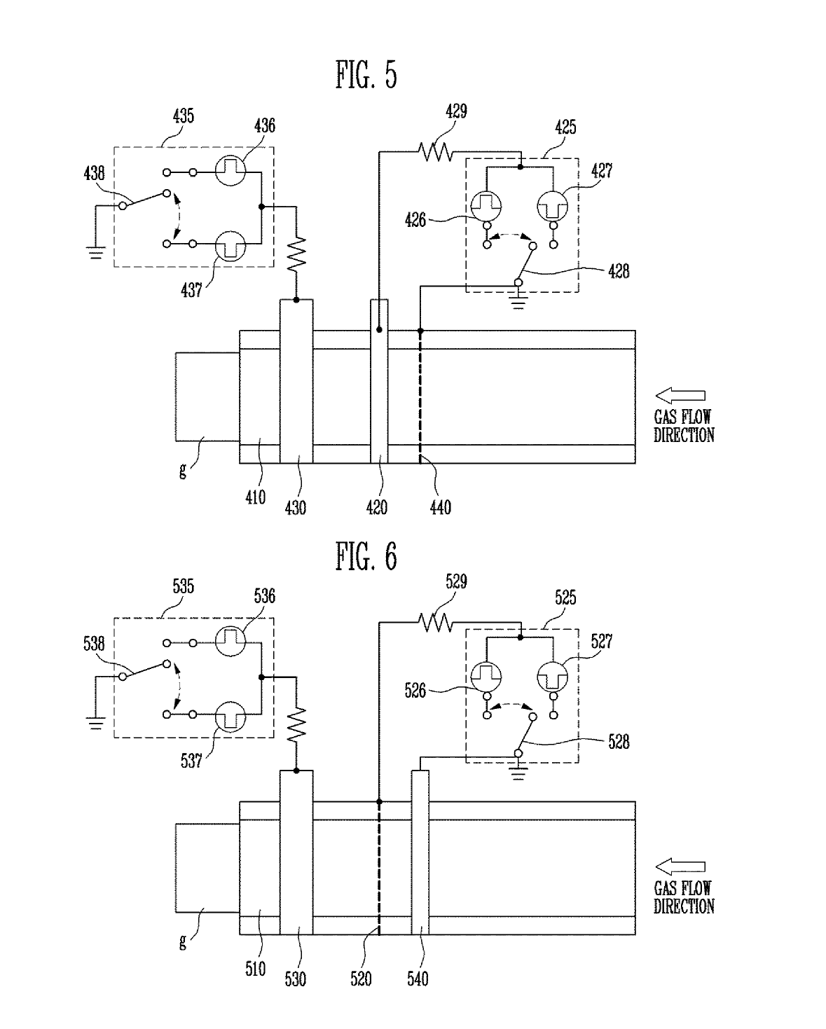

FIGS. 5 and 6 are views for explaining a pulse power source in a plasma system according to another embodiment of the present disclosure. Referring to FIG. 5, the plasma system includes a nozzle 410, first electrode 420, second electrode 430, and third electrode 440, and a structure and shape thereof are very similar to that of the nozzle 210, first electrode 220, second electrode 230, and third electrode 240, respectively, and thus further explanation is omitted.

The first electrode 420 is connected to a first power source 425 through a first resistance 429. The first power source 425 includes a first pulse generator 426, second pulse generator 427, and switch 428. The first pulse generator 426 generates a positive pulse voltage, and the second pulse generator 427 generates a negative pulse voltage. The switch 428 may be connected to the first pulse generator 426 or the second pulse generator 427 by an external control signal, or not connected to any of them. When the switch 428 is connected to the first pulse generator 426, the first power source 425 outputs a positive pulse voltage to the first electrode 420, and when the switch 428 is connected to the second pulse generator 427, the first power source 425 outputs a negative pulse voltage to the first electrode 420. The second power source 435 is very similar to the first power source 425, and thus further explanation is omitted.

The first electrode 420 and second electrode 420 electrically connected to the first power source 425 and second power source 435, respectively, may operate as either an anode or cathode depending on an operation of the switch 428, 438.

Referring to FIG. 6, a plasma system includes a nozzle 510, first electrode 520, second electrode 530, and third electrode 540, and a structure and shape thereof are very similar to that of the nozzle 310, first electrode 320, second electrode 330, and third electrode 340, respectively, and thus further explanation is omitted. In the embodiment illustrated in FIG. 5, the first electrode 420 is formed on a portion of the outer circumference (o) and the third electrode 440 is formed on a portion of the inner circumference (i). However, in the embodiment illustrated in FIG. 6, the first electrode 520 is formed on a portion of the inner circumference (i) and the third electrode 540 is formed on a portion of the outer circumference (o). The first power source 525 and second power source 535 are very similar to the first power source 425 and second power source 435, respectively, and thus further explanation is omitted.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A plasma system comprising:
a nozzle comprising an outer circumference exposed towards outside, an inner circumference facing the outer circumference and touching gas, and an exit from which the gas is sprayed;
a first electrode formed on a portion of the outer circumference or inner circumference; and
a second electrode formed on a portion of the outer circumference and distanced from the first electrode;
wherein the first electrode is electrically connected to a first power source having a first voltage, and the second electrode is electrically connected to a second power source having a second voltage that is different from the first voltage, and
the second electrode is formed closer to the exit than the first electrode.

2. The plasma system according to claim 1,
further comprising a third electrode formed on a surface facing the surface where the first electrode is formed and distanced from the second electrode, the third electrode being electrically connected to a ground of the first power source.

3. The plasma system according to claim 2,
wherein a distance between the third electrode and the exit is shorter than a distance between the first electrode and the exit, but longer than a distance between the second electrode and the exit.

4. The plasma system according to claim 2,
wherein a distance between the third electrode and the exit is longer than a distance between the first electrode and the exit and a distance between the second electrode and the exit.

5. The plasma system according to claim 1,
wherein the first electrode is electrically connected to the first power source through a first resistance, and the second electrode is electrically connected to the second power source through a second resistance.

6. The plasma system according to claim 5,
wherein the first resistance and second resistance are ballast resistances.

7. The plasma system according to claim 1,
wherein the first power source and second power source comprises a direct current power source or alternative current power source.

8. The plasma system according to claim 1,
wherein the first power source and second power source each comprises a pulse power source.

9. The plasma system according to claim 8,
wherein the pulse power source comprises a first pulse generator configured to generate a positive pulse voltage;
a second pulse generator configured to generate a negative pulse voltage; and
a switch electrically connected to the first pulse generator or second pulse generator,
wherein in response to the switch being electrically connected to the first pulse generator, the pulse power source outputs the positive pulse voltage, and in response to the switch being electrically connected to the second pulse generator, the pulse power source outputs the negative pulse voltage.

10. The plasma system according to claim 1,
wherein the nozzle is made of an insulator material, and
the inner circumference of the nozzle is processed to be round.
